(19) United States
(12) Patent Application Publication
(10) Pub. No.: US 2015/0307540 A1
(43) Pub. Date: Oct. 29, 2015

(54) AMORPHOUS FORM OF DAPAGLIFLOZIN 1,2-PROPANEDIOL

(71) Applicant: CADILA HEALTHCARE LIMITED, Ahmedabad (IN)

(72) Inventors: Shri Prakash Dhar DWIVEDI, Vadodora (IN); Brij KHERA, Ahmedabad (IN); Jagdish Maganlal PATEL, Vadodora (IN); Sanjay Jagdish DESAI, Vadodora (IN); Jayprakash Ajitsingh PARIHAR, Vadodora (IN); Mahesh Laljibhai RUPAPARA, Vadodora (IN)

(73) Assignee: CADILA HEALTHCARE LIMITED, Ahmedabad (IN)

(21) Appl. No.: 14/626,341

(22) Filed: Feb. 19, 2015

(30) Foreign Application Priority Data
Feb. 21, 2014 (IN) 626/MUM/2014

(51) Int. Cl. C07H 7/04; C07C 31/20

(52) U.S. Cl. CPC C07H 7/04; C07C 31/205

(57) ABSTRACT

The invention provides an amorphous form of dapagliflozin 1,2-propanediol of Formula (A) or hydrates thereof and their process for preparation. The present invention also provides a pharmaceutical composition comprising art amorphous solid dispersion containing dapagliflozin 1,2-propanediol or hydrates thereof.

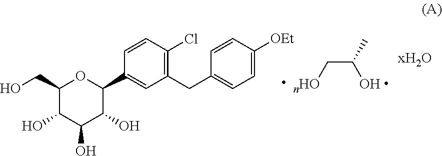

### FIELD OF THE INVENTION

The invention relates to an amorphous form of dapagliflozin 1,2-propanediol. In particular, the present invention relates to an amorphous form of dapagliflozin 1,2-propanediol or hydrates thereof and their process for preparation.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

FARXIGA® is a sodium-glucose cotransporter 2 (SGLT2) inhibitor indicated as an adjunct to diet and exercise to improve glycemic control in adults with type 2 diabetes mellitus. The active ingredient of the approved product FARXIGA Dapagliflozin, which is described chemically as D-glucitol, (1S)-1,5-anhydro-1-C-[4-chloro-3-[(4-ethoxyphenyl)methyl]phenyl] compounded with (2S)-1,2-propanediol, hydrate (1:1:1). The empirical formula is C₂₁H₂₅ClO₆.C₃H₈O₂.2H₂O and the molecular weight is 502.98. The structural formula is:

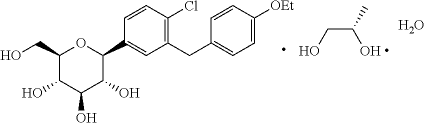

U.S. Pat. No. 6,515,117 B2 discloses the compound dapagliflozin and provides its process for preparation.

WO 2004/002824 relates to crystalline forms and solvates of (1S)-1,5-anhydro-1-C-[3((phenyl)methyl)phenyl)-D-glucitol derivatives and their complexes with amino acids. In particular, it discloses crystalline polymorphs of dapagliflozin, for example in the form of a propylene glycol hydrate.

WO 2008/116178 refers to pharmaceutical formulations which include crystalline dapagliflozin propylene glycol hydrate.

WO 2012/163546 discloses pharmaceutical compositions comprising dapagliflozin and cyclodextrin in the form of inclusion bodies.

U.S. Pat. No. 7,919,598; U.S. 2013/0303467 A1 and WO 2013/079501 A1 describe various crystalline forms of dapagliflozin viz. hydrates, anhydrous forms, solvates and complexes with amines and amino acids.

U.S. 2013/0237487 A1 discloses an amorphous form of dapagliflozin.

WO 2015/011113 discloses an amorphous solid dispersion comprising at least one polymer and dapagliflozin and a pharmaceutical composition comprising said amorphous solid dispersion and the process for the preparation thereof.

The prior-arts disclose one or the other crystalline form of dapagliflozin or 1,2-propanediol hydrate or solvates thereof or an amorphous form of dapagliflozin and an amorphous solid dispersion comprising dapagliflozin and at least one polymer. None of them provide amorphous form of the approved drug candidate dapagliflozin 1,2-propanediol or hydrates thereof. In view of the above art, there is provided an amorphous form of the approved drag candidate dapagliflozin 1,2-propanediol or hydrates thereof.

### SUMMARY OF THE INVENTION

In one general aspect, there is provided an amorphous form of dapagliflozin 1,2-propanediol or hydrates thereof of Formula (A),

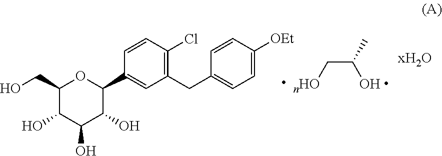

wherein n is 0.8 to 1.2 and x is 0-2.

In another general aspect, there is provided a process for the preparation of an amorphous form of dapagliflozin 1,2-propanediol or hydrates thereof of Formula (A).

In another general aspect, there is provided a process for the preparation of an amorphous form of dapagliflozin 1,2-propanediol or hydrates thereof of Formula (A) using spray drying.

In another general aspect, there is provided a process for the preparation of an amorphous form of dapagliflozin 1,2-propanediol or hydrates thereof by temperature alterations of dapagliflozin 1,2-propanediol or hydrates thereof in presence or absence of one or more solvents.

In another general aspect, there is provided a process for the preparation of an amorphous dapagliflozin 1,2-propanediol or hydrates, the process comprising:
(a) providing a solution or suspension of dapagliflozin 1,2-propanediol or hydrates thereof in presence of one or more excipients in one or more solvents; and
(b) obtaining the amorphous dapagliflozin 1,2-propanediol or hydrates thereof by the removal of the solvents.

In another general aspect, there is provided an amorphous solid dispersion comprising dapagliflozin 1,2-propanediol or hydrate thereof and one or more pharmaceutically acceptable excipients.

In another general aspect, there is provided a pharmaceutical composition containing an amorphous form of dapagliflozin 1,2-propanediol or hydrates thereof, optionally with one or more pharmaceutically acceptable earners and one or more pharmaceutically acceptable excipients.

In another general aspect, there is provided a pharmaceutical composition comprising an amorphous solid dispersion comprising dapagliflozin 1,2-propanediol or hydrates thereof together with one or more pharmaceutically acceptable carriers, excipients or diluents.

### BRIEF DESCRIPTION OF DRAWINGS

FIG. 1: x-ray powder diffractogram (XRPD) of amorphous dapagliflozin 1,2-propanediol prepared in example-1.

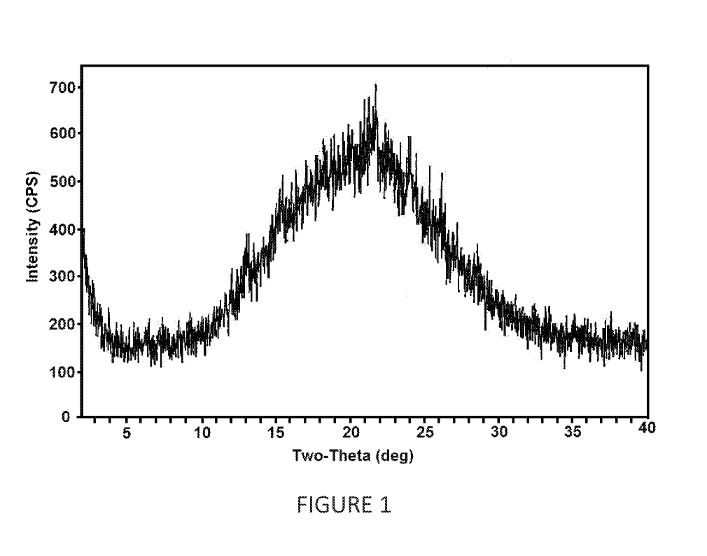

FIG. 2: x-ray powder diffractogram (XRPD) of amorphous dapagliflozin 1,2-propanediol prepared in example-3.

### DETAILED DESCRIPTION OF THE INVENTION

The aforementioned objectives of the present invention are fulfilled by one or more of the processes described herein.

All ranges recited herein include the endpoints, including those that recite a range “between” two values. Terms such as “about”, “from”, and “to” are to be construed as modifying a term or value such that it is not an absolute. This includes, at very least, the degree of expected experimental error, technique error and instrument error for a given technique used to measure a value.

As used herein, the terms “suspending” may be interchanged with “slurring” or “triturating”, and refer to a process earned out in a heterogeneous mixture where complete dissolution does not occur. Also, heating the suspension or slurry can result in a homogenous mixture where complete or partial dissolution occurs at an elevated temperature or ambient temperature.

As used herein, the term “solution” does not limit to a clear solution only and includes a hazy solution or slurry which is a heterogeneous mixture.

As used herein, the term “temperature alterations” means change of temperature which includes increasing or decreasing the temperature.

The term “pharmaceutical compositions” herein includes pharmaceutical formulations like tablets, pills, powders, liquids, suspensions, emulsions, granules, capsules, suppositories, or injection preparations.

The term “composition” used herein means a physical mixture of two or more components.

As used herein, the terms “obtaining” means isolating the amorphous form of dapagliflozin 1,2-propanediol or hydrates by way of filtration, filtration under vacuum, centrifugation, and decantation. The product obtained may be further or additionally dried to achieve the desired moisture values. For example, the product may be dried in a tray drier, dried under vacuum and/or in a Fluid Bed Drier.

As used herein, the term “solid dispersion” means any solid composition having at least two components. In certain embodiments, a solid dispersion as disclosed herein includes an active ingredient dapagliflozin 1,2-propanediol or hydrates thereof dispersed among at least one other component, for example a polymer.

The term “immobilize” as used herein with reference to the immobilization of the active compound i.e., dapagliflozin 1,2-propanediol or hydrates thereof in the polymer matrix, means that molecules of the active compound interact with molecules of the polymer in such a way that the molecules of the dapagliflozin 1,2-propanediol or hydrates thereof are held in the aforementioned matrix and prevented from crystal nucleation due to lack of mobility.

In one general aspect there is provided an amorphous form of dapagliflozin 1,2-propanediol or hydrates thereof of Formula (A),

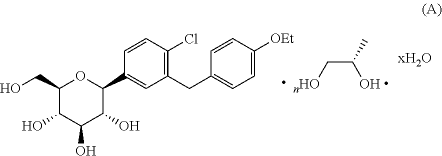

wherein n is 0.8 to 1.2 and x is 0-2.

In another general aspect, there is provided an amorphous form of dapagliflozin 1,2-propanediol or hydrates thereof having purity by HPLC of greater than about 99%.

In general, the amorphous form of dapagliflozin 1,2-propanediol or hydrates thereof is having residual solvents within the permissible ICH limits suitable for pharmaceutical preparations. For example 1,2-propandiol is present within the permissible ICH limits suitable for pharmaceutical preparations.

The amorphous form of dapagliflozin 1,2-propanediol or hydrates thereof of Formula (A) of the present invention, contains 1,2-propanediol content from about 10 to 30%; in particular from about 12 to 25%, more particularly from about 15 to 18%.

The amorphous form of dapagliflozin 1,2-propanediol or hydrates thereof of Formula (A) of the present invention is having a water content of up to about 8% wt/wt.

The amorphous form of dapagliflozin 1,2-propanediol or hydrates thereof of Formula (A) of the present invention is about 1:1 composition of dapagliflozin and 1,2-propanediol containing xH₂O as water content, wherein x is 0 to 2.

In another general aspect, there is provided a process for the preparation of an amorphous form of dapagliflozin 1,2-propanediol or hydrates, the process comprising:
(a) providing a solution or suspension of dapagliflozin 1,2-propanediol or hydrates thereof in one or more of solvents; and
(b) obtaining the amorphous form of dapagliflozin 1,2-propanediol or hydrates by removal of the solvents.

The step (a) above involves providing a solution or suspension of dapagliflozin 1,2-propanediol or hydrates in one or more of solvents or mixture thereof.

The solution or suspension for step (a) can be obtained by known methods that include:
(i) direct use of a reaction mixture containing dapagliflozin 1,2-propanediol or hydrates that is obtained in the course of its synthesis; or
(ii) dissolving dapagliflozin 1,2-propanediol or hydrates in one or more of solvents or mixture thereof.

In general, in step (a) any physical form of dapagliflozin 1,2-propanediol or hydrates thereof may be utilized for providing the solution of dapagliflozin 1,2-propanediol or hydrates thereof in one or more of solvents or mixture thereof. The dissolution temperatures may be from about below 0° C. to about the reflux temperature of the solvent.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the C₁₋₄ alcohol is selected from methanol, ethanol, n-propanol, isopropanol and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

The step (b) above involves obtaining of an amorphous form of dapagliflozin 1,2-propanediol or hydrates thereof from the solution or suspension of step (a). The isolation of an amorphous form of dapagliflozin 1,2-propanediol or hydrates thereof may be affected by removing the solvents. The techniques which may be used for the removal of solvents comprises one or more of distillation, distillation under vacuum, spray drying, agitated thin film drying (“ATFD”), freeze drying (lyophilization), filtration, decantation, and centrifugation. The solvent may also be removed, optionally, at reduced pressure and/or at elevated temperature.

In general, freeze drying (lyophilization) may be performed by freezing a solution or suspension of dapagliflozin 1,2-propanediol or hydrates at low temperatures and reducing the pressure to remove the solvents from the frozen solution of dapagliflozin 1,2-propanediol or hydrates. Temperatures that may be required to freeze the solution, depending on the solvent chosen to make the solution of dapagliflozin 1,2-propanediol or hydrates may range from about −70° C. to about 10° C.

In another general aspect, there is provided a process for preparation of amorphous form of dapagliflozin 1,2-propanediol or hydrates thereof by spray drying the solution of dapagliflozin 1,2-propanediol or hydrates thereof in one or more solvents.

In another general aspect, there is provided a process for preparation of an amorphous form of dapagliflozin 1,2-propanediol or hydrates thereof of Formula (A), comprising spray drying a solution of dapagliflozin and 1,2-propanediol. In general, the process involves spray drying of the feed stock.

In general, the preferred aspect of the invention involves spray drying of feed stock which is prepared as discussed herein below, wherein any solid forms of dapagliflozin 1,2-propanediol or hydrates thereof may be used. In particular, the spray drying of dapagliflozin 1,2-propanediol may be performed maintaining the inlet temperature in the range of 35° C.-80° C., nitrogen pressure of 2-6 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

Any known form of dapagliflozin 1,2-propanediol or the filtered cake that is obtained as an end result of the reaction, or reaction mass comprising dapagliflozin 1,2-propane diol or hydrates thereof or solution comprising dapagliflozin and 1,2-propanediol can be used as input for the preparation of feed stock.

In the present invention, feed stock of dapagliflozin 1,2-propanediol of Formula (A) is conveniently prepared by dissolving dapagliflozin obtained as per U.S. Pat. No. 6,515,117 B2 (as described in Example G) and 1,2-propanediol in one or more solvents. The solvents is selected from the group comprising one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In particular, methanol, ethanol, acetone, ethyl acetate, methylene dichloride, water-methanol, water-ethanol, water-acetone or mixture of solvents is used or such solvents that evaporate easily to afford dry product. Most particularly, acetone, methanol, ethanol, ethyl acetate, methylene dichloride or mixtures thereof may be used.

According to further general aspect, any form of dapagliflozin 1,2-propanediol or hydrates thereof can be spray dried by dissolving or suspending or slurring in one or more solvents or solvent-water system to get amorphous form. In the present invention, feed stock of dapagliflozin 1,2-propanediol in methanol is spray-dried. Thus obtain spry-dried compound is in amorphous form.

In another general aspect, there is provided a process for preparation of amorphous form of dapagliflozin 1,2-propanediol or hydrates thereof of Formula (A) by temperature alterations of dapagliflozin 1,2-propanediol or hydrates thereof of Formula (A) in presence or absence of one or more solvents.

In another general aspect, there is provided a process for the preparation of an amorphous form of dapagliflozin 1,2-propanediol or hydrates thereof of Formula (A),

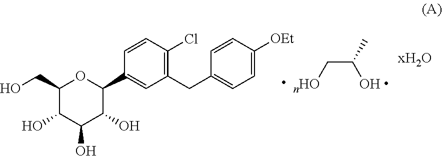

wherein n is 0.8 to 1.2 and x is 0-2.
the process comprising:
(a) heating dapagliflozin 1,2-propanediol or hydrates thereof of Formula (A), optionally in the presence of one or more excipients in one or more solvents at a first temperature; and
(b) cooling to obtain the amorphous form of dapagliflozin 1,2-propanediol or hydrates thereof to a second temperature.

The first temperature herein is higher than the second temperature. The difference in the amplitude between the first and the second temperatures may be at least about 20° C. In particular, about 30° C., or more particularly about 50° C. In general, the first temperature is from about 50° C. to about 150° C. and the second temperature is from about 0° C. to about 35° C.

In general, dapagliflozin 1,2-propanediol of Formula (A) may be heated optionally in the presence of one or more solvents at first temperature and then cooled to a second temperature to obtain the amorphous form of dapagliflozin 1,2-propanediol of Formula (A).

In general, dapagliflozin 1,2-propanediol or hydrates thereof may be heated to a first temperature which may be less than or equal to its melting point, or optionally higher than the melting point and cooled to a second temperature which is lower than the first temperature to obtain the amorphous form of dapagliflozin 1,2-propanediol or hydrates thereof of Formula (A).

In another general aspect, there is provided an amorphous solid dispersion comprising dapagliflozin 1,2-propanediol or hydrates thereof and one or more pharmaceutically acceptable excipients. The pharmaceutically acceptable excipient is a non-ionic polymer or an ionic polymer.

In general, the amorphous solid dispersion of dapagliflozin 1,2-propanediol or hydrates thereof having a purity by HPLC of more than about 99%.

In general, the polymer is selected from methacrylic acid copolymers; polyvinylpyrrolidone (PVP), 4-vinylpyrrolidone-vinyl acetate copolymer (copovidone) or copolymers of methacrylic acid and ethylacrylate (EUDRAGIT® L100-55), hydroxy-propyl cellulose, hydroxypropylmethyl cellulose (HPMC), hypromellose phthalate, or hydroxypropylmethyl cellulose acetate succinate (HPMC-AS). In particular, PVP of different grades like K-15, K-30, K-60, K-90 and K-120 may be used for the preparation of amorphous solid dispersion. More particular, hydroxypropylmethyl cellulose (HPMC) or hydroxypropylmethyl cellulose acetate succinate (HPMC-AS) and PVP K-30 may be used.

In another general aspect, there is provided an amorphous solid dispersion comprising dapagliflozin 1,2-propanediol or hydrates thereof and one or more pharmaceutically acceptable excipients.

In some embodiments, the dapagliflozin 1,2-propanediol or hydrates thereof of Formula (A) may be dispersed within a matrix formed by a polymer in its solid state such that it is immobilized in its amorphous form. The polymer may prevent intramolecular hydrogen bonding or weak dispersion forces between two or more drug molecules of dapagliflozin 1,2-propanediol.

In some embodiments, the ratio of the amount of weight of dapagliflozin 1,2-propanediol within the solid dispersion to the amount by weight of the polymer therein is from about 1:1 to about 1:10. The composition of dapagliflozin 1,2-propanediol with polymer may be prepared by using about 1:1 to about 1:10 polymers with respect to dapagliflozin 1,2-propanediol.

In another general aspect, there is provided a process for the preparation of an amorphous solid dispersion comprising dapagliflozin 1,2-propanediol or hydrates thereof and one or more pharmaceutically acceptable excipients, the process comprising:
(a) providing a solution of dapagliflozin 1,2-propanediol or hydrates thereof and one or more pharmaceutically acceptable excipients in one or more solvents; and
(b) obtaining the amorphous dapagliflozin 1,2-propanediol or hydrates thereof by the removal of the solvents.

In general, a solution of dapagliflozin 1,2-propanediol or hydrates thereof in presence of one or more excipients in one or more solvents is obtained by the known methods that include:
(i) direct use of a reaction mixture containing dapagliflozin 1,2-propanediol that is obtained in the course of its synthesis and addition of excipients; or
(ii) dissolving dapagliflozin 1,2-propanediol and excipients in one or more solvents.

In general, in step a) any physical form of dapagliflozin 1,2-propanediol may be utilized for providing the solution of dapagliflozin 1,2-propanediol in one or more solvents. The dissolution temperatures may be from 0° C. to the reflux temperature of the solvent. In particular, the dissolution may be performed from 25° C. to 120° C., so as to obtain the clear solution of dapagliflozin 1,2-propanediol.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the C₁₋₄ alcohol is selected from methanol, ethanol, n-propanol, isopropanol, and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

In general, the excipients comprises of non-ionic polymer or an ionic polymer. In particular, the polymer is selected from methacrylic acid copolymers, polyvinylpyrrolidone (PVP), 4-vinylpyrrolidone-vinyl acetate copolymer (copovidone) or copolymers of methacrylic acid and ethylacrylate (EUDRAGIT® L100-55), hydroxypropyl cellulose, hydroxypropylmethyl cellulose (HPMC), hypromellose phthalate, or hydroxypropylmethyl cellulose acetate succinate (HPMC-AS). In particular, PVP of different grades like K-15, K-30, K-60, K-90 and K-120 may be used to prepare the feed stock. More particular, hydroxypropylmethyl cellulose (HPMC) or its acetate succinate and PVP K-30 may be used. HPMC with viscosity 8 cps, 5 cps or 3 cps may be used.

In some embodiments, the ratio of the amount of weight dapagliflozin 1,2-propanediol of Formula (A) and the amount by weight of the excipient is from about 1:1 to about 1:10. The solution of dapagliflozin 1,2-propanediol in presence of excipient in one or more solvents, preferably PVP K-30 or HPMC-AS may be prepared by using about 1:1 to about 1:10 polymers with respect to dapagliflozin 1,2-propanediol.

The step b) involves removal of the solvent to obtain an amorphous dapagliflozin 1,2-propanediol. The isolation may be affected by removing solvents. Techniques which may be used for the removal of solvents include distillation, distillation under vacuum, spray drying, agitated thin film drying (“ATFD”), and freeze drying (lyophilization).

The solvent may be removed, optionally under reduced pressures, at temperatures less than 70° C., less than 60° C., less than 50° C.

In general, freeze drying (lyophilization) may be performed by freezing a solution of dapagliflozin 1,2-propanediol optionally in presence of one or more excipients at low temperatures and reducing the pressure to remove the solvents from the frozen solution of dapagliflozin 1,2-propanediol. Temperatures that may be required to freeze the solution, depending on the solvents chosen to make the solution of dapagliflozin 1,2-propanediol may range from −70° C. to 10° C.

In general, the preferred aspect of the invention involves spray drying of dapagliflozin 1,2-propanediol solution comprises of spray drying of feed stock, which is prepared as discussed below, wherein any solid forms of dapagliflozin 1,2-propanediol or hydrates thereof in presence of one or more excipients is used. In particular, the spray drying of dapagliflozin 1,2-propanediol or hydrates thereof in presence of excipients may be performed maintaining the inlet temperature in the range of 35° C.-80° C., nitrogen pressure of 2-6 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

In another general aspect, the present invention provides an amorphous form of dapagliflozin 1,2-propanediol or hydrates thereof having purity by HPLC of>99%. In particular, the purity by HPLC of>99.5%, more particularly, the purity by HPLC of>99.8%, most particularly, the purity by HPLC>99.9%.

In another general aspect, the present invention provides an amorphous solid dispersion of dapagliflozin 1,2-propanediol or hydrates thereof having purity by HPLC of>99%. In particular, the purity by HPLC of>99.5%, more particularly, the purity by HPLC of>99.8%, most particularly, the purity by HPLC>99.9%.

The invention also encompasses a pharmaceutical compositions containing an amorphous form of dapagliflozin 1,2-propanediol or hydrates thereof. As used herein, the term “pharmaceutical compositions” includes pharmaceutical formulations comprises one or more of tablets, pills, powders, liquids, suspensions, emulsions, granules, capsules, suppositories, and injection preparations.

In another general aspect, there is provided a pharmaceutical composition containing an amorphous form of dapagliflozin 1,2-propanediol or hydrates thereof, optionally with one or more pharmaceutically acceptable carriers and one or more pharmaceutically acceptable excipients.

In another general aspect, there is provided a pharmaceutical composition comprising an amorphous solid dispersion containing dapagliflozin 1,2-propanediol or hydrates thereof together with one or more pharmaceutically acceptable carriers, excipients or diluents.

In general, the pharmaceutical compositions containing the dapagliflozin 1,2-propanediol or hydrates of the invention may be prepared by using diluents or excipients selected from fillers, bulking agents, binders, wetting agents, disintegrating agents, surface active agents, and lubricants.

Various modes of administration of the pharmaceutical compositions of the invention is selected depending on the therapeutic purpose, for example tablets, pills, powders, liquids, suspensions, emulsions, granules, capsules, suppositories, or injection preparations.

The present invention is further illustrated by following examples which are provided merely to exemplify the invention and do not limit the scope of it.

### EXAMPLES

### Example-1

### Preparation of Amorphous Dapagliflozin 1,2-Propanediol by Spray Drying

Dapagliflozin (5 g), 1,2-propanediol (1 g) and methanol (100 mL) were taken into a round bottom flask. The content was stirred for 1 hour at 55-60° C. The content was filtered through hyflosupercel and washed with 10.0 mL methanol. The clear filtrate was subjected to spray drying in JISL Mini spray drier LSD-48 by maintaining the inlet temperature in the range of 50-55° C., under nitrogen pressure of 4.0 kg/cm² at a feed rate of 12%, to obtain amorphous dapagliflozin 1,2-propanediol. [1,2-propanediol content (By GC): 18%].

### Example-2

### Preparation of Amorphous Dapagliflozin 1,2-Propanediol by Temperature Alterations

Dapagliflozin 1,2-propanediol hydrate (5 g) was heated to 90-105° C. on a hot plate and cooled to 10-15° C. to obtain amorphous dapagliflozin 1,2-propanediol.

### Example-3

### Preparation of Amorphous Dapagliflozin 1,2-Propanediol by Spray Drying in presence of excipients

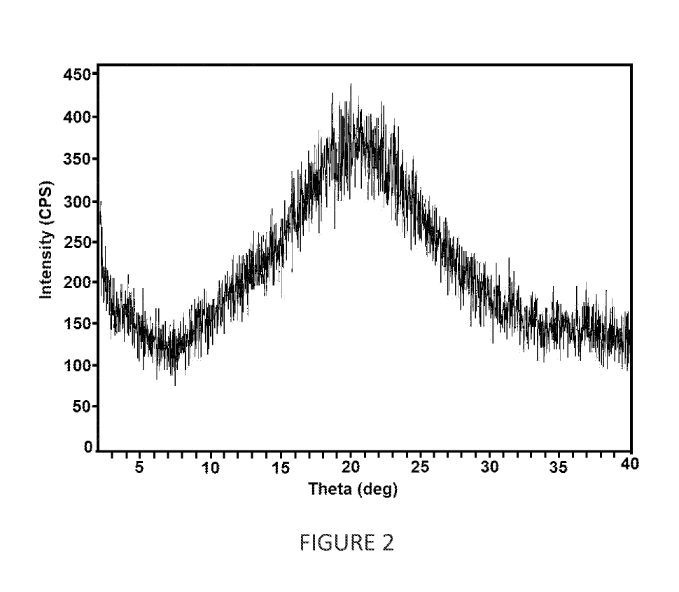

Dapagliflozin (5 g), 1,2-propanediol (1 g) and mixture of methanol and methylene dichloride (75 mL) were taken in round bottom flask at 25° C. to 30° C. The reaction mixture was stirred for 1 hour at 55-60° C. HPMC-AS (3 cps) (2.5 g) and in mixture of methanol and methylene dichloride (25 mL) were added to the reaction mixture and stirred. The solution thus obtained was spray dried in a clean LU-222 Advanced model (twin cyclone) spray dryer having inlet air temperature at 60° C., outlet temperature at 50° C., air pressure at 4 Kg cm², aspirator-blower at 99 RPM, initial vacuum of 100 mmHg and peristaltic pump at 11 RPM. The product was collected from cyclone and was further dried at to get 2.85 g of amorphous dapagliflozin 1,2-propanediol characterized by x-ray powder diffraction pattern (FIG. 2).

### Example-4

### Preparation of Amorphous Dapagliflozin 1,2-Propanediol by Temperature Alterations

Dapagliflozin 1,2-propanediol hydrate (5 g) and 1.25 g HPMC-AS (3 cps) was heated to 90-105° C. on a hot plate and cooled to 10-15° C. to obtain amorphous dapagliflozin 1,2-propanediol.

### Example-5

### Preparation of amorphous solid dispersion of dapagliflozin 1,2-propanediol with polyvinylpyrrolidone

Dapagliflozin 1,2-propanediol hydrate (5 g) was added to methanol (50 mL) at 25-30° C. and the contents were stirred for 5 minutes at the same temperature, followed by heating at 50° C. to form a clear solution. The resulting solution was cooled to room temperature (25-35° C.) and then polyvinylpyrrolidone (2.5 g) was added at the same temperature to obtain a clear solution. The resulting solution was stirred for 30 minutes at room temperature, followed by the removal of solvent by distillation under vacuum at 65-70° C. to obtain 5.5 g amorphous solid dispersion of dapagliflozin 1,2-propanediol with polyvinylpyrrolidone.

While the present invention has been described in terms of its specific embodiments, certain modifications and equivalents will be apparent to those skilled in the art and are intended to be included within the scope of the present invention.

1. An amorphous form of dapagliflozin 1,2-propanediol or hydrates thereof of Formula (A), 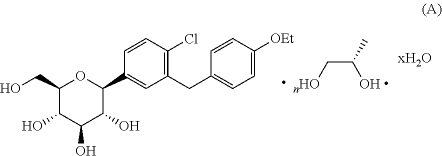
wherein n is 0.8 to 1.2 and x is 0-2.

2. The amorphous form of dapagliflozin 1,2-propanediol or hydrates thereof according to claim 1 having a purity by HPLC of greater than about 99%.

3. The amorphous form of dapagliflozin 1,2-propanediol or hydrates thereof according to claim 1 having a water content of up to about 8% wt/wt.

4. The amorphous form of dapagliflozin 1,2-propanediol or hydrates thereof according to claim 1 is about 1:1 composition of dapagliflozin and 1,2-propanediol containing xH₂O as water content, wherein x is 0 to 2.

5. A process for the preparation of an amorphous form of dapagliflozin 1,2-propanediol or hydrates thereof according to claim 1, the process comprising:
(a) providing a solution or suspension of dapagliflozin 1,2-propanediol or hydrates thereof in one or more solvents; and
(b) obtaining the amorphous form of dapagliflozin 1,2-propanediol or hydrates by the removal of the solvents.

6. The process according to claim 5, wherein the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane, or mixtures thereof.

7. The process according to claim 6, wherein the C₁₋₄ alcohol is selected from methanol, ethanol, n-propanol, isopropanol, and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone.

8. The process according to claim 5, wherein the removal of the solvent comprises one or more of distillation, distillation under vacuum, spray drying, agitated thin film Gyring (“ATFD”), freeze drying (lyophilization), filtration, decantation, and centrifugation.

9. A process for the preparation of an amorphous form of dapagliflozin 1,2-propanediol or hydrates thereof of Formula (A), 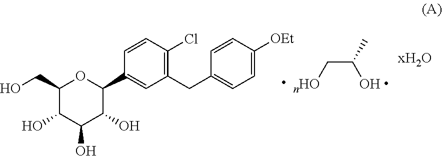
wherein n is 0.8 to 1.2 and x is 0-2,
the process comprising:
(a) heating dapagliflozin 1,2-propanediol or hydrates thereof of Formula (A), optionally in the presence of one or more excipients in one or more solvents at a first temperature; and
(b) cooling to obtain the amorphous form of dapagliflozin 1,2-propanediol or hydrates thereof to a second temperature.

10. The process according to claim 9, wherein the first temperature is from about 50° C. to about 150° C.

11. The process according to claim 9, wherein the second temperature is from about 0° C. to about 150° C.

12. The process according to claim 9, wherein the excipient is a non-ionic polymer or an ionic polymer selected from methacrylic acid copolymers, polyvinylpyrrolidone (PVP), 4-vinylpyrrolidone-vinyl acetate copolymer (copovidone) or copolymers of methacrylic acid and ethylacrylate (EUDRAGIT® L100-55), hydroxy-propyl cellulose, hydroxypropylmethyl cellulose (HPMC), hypromellose phthalate, or hydroxypropylmethyl cellulose acetate succinate (HPMC-AS).

13. An amorphous solid dispersion comprising dapagliflozin 1,2-propanediol or hydrates thereof and one or more pharmaceutically acceptable excipients.

14. The amorphous solid dispersion of dapagliflozin 1,2-propanediol or hydrates thereof according to claim 13 having a purity by HPLC of more than about 99%.

15. A process for the preparation of an amorphous solid dispersion comprising dapagliflozin 1,2-propanediol or hydrates thereof according to claim 13, and one or more pharmaceutically acceptable excipients, the process comprising:
(a) providing a solution of dapagliflozin 1,2-propanediol or hydrates thereof and one or more pharmaceutically acceptable excipients in one or more solvents; and
(b) obtaining the amorphous solid dispersion of dapagliflozin 1,2-propanediol or hydrates thereof with the pharmaceutically acceptable excipients by the removal of the solvents.

16. The process according to claim 15, wherein the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane, or mixtures thereof.

17. The process according to claim 15, wherein the C₁₋₄ alcohol is selected from methanol, ethanol, n-propanol, isopropanol, and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone.

18. The amorphous solid dispersion according to claim 15, wherein the pharmaceutically acceptable excipient is a non-ionic polymer or an ionic polymer.

19. The amorphous solid dispersion according to claim 15, wherein the polymer is selected from methacrylic acid copolymers, polyvinylpyrrolidone (PVP), 4-vinylpyrrolidone-vinyl acetate copolymer (copovidone) or copolymers of methacrylic acid and ethylacrylate (EUDRAGIT® L100-55), hydroxypropylcellulose, hydroxypropylmethyl cellulose (HPMC), hypromellose phthalate, or hydroxypropylmethyl cellulose acetate succinate (HPMC-AS).

20. A pharmaceutical composition containing an amorphous form of dapagliflozin 1,2-propanediol or hydrates thereof according to claim 1, optionally with one or more pharmaceutically acceptable carriers and one or more pharmaceutically acceptable excipients.

21. A pharmaceutical composition comprising an amorphous solid dispersion containing dapagliflozin 1,2-propanediol or hydrates thereof according to claim 1, together with one or more pharmaceutically acceptable carriers, excipients or diluents.
